(19) United States
(12) Patent Application Publication
(10) Pub. No.: US 2011/0268777 A1
(43) Pub. Date: Nov. 3, 2011

(54) Lotion Composition for Wet Wipes

(76) Inventors: Randall Glenn Marsh, Hamilton, OH (US); Thomas James Klofta, Cincinnati, OH (US); Kristin Hofmann Miller, Springboro, OH (US); Jacqueline Marie Duderstadt, Cincinnati, OH (US)

(21) Appl. No.: 12/771,391

(22) Filed: Apr. 30, 2010

(51) Int. Cl. A61K 8/02; A61K 8/97; A61K 8/37; A61Q 19/00

(52) U.S. Cl. 424/401; 424/725; 514/549

(57) ABSTRACT

A lotion composition for a skin cleaning wipe comprises a superwetter and a rheology modifier, adapted to provide an enhanced sensation of wetness with a reduced quantity of liquid. Complementary emollients, emulsifiers, and adjuncts and a method for increasing the wetness perception of a wet wipe are also described.

### FIELD OF THE INVENTION

The disclosure relates to lotion compositions for personal care wipes, and more particularly to low pH compositions having improved sensorial properties related to wetness and skin softness.

### BACKGROUND OF THE INVENTION

Personal care wipes, such as baby wipes, facial cleansing wipes, intimate cleansing wipes, flushable moist wipes, and the like, typically comprise a nonwoven substrate and a lotion composition. The lotion composition may wet the substrate to facilitate cleaning. The lotion composition may also comprise ingredients to soothe, soften, or care for the skin, to improve the feel of the lotion, to improve the removal of residues from the skin, to provide pleasant scents, to prevent bacterial growth, etc.

Some lotion compositions have a pH at or near 5.5. A pH at or near 5.5 is close to physiological skin pH. Low pH lotion compositions may have a pH at or near 3.8. Low pH lotion compositions may provide at least two advantages over typical lotion compositions. In cases where a wipe is being used to remove alkaline residues, such as residues from a bowel movement, a low pH lotion composition may help restore a healthy, acidic skin pH of approximately 5 and/or render irritants from fecal matter non-irritating, as by inactivating fecal enzymes. Low pH lotions may also inhibit microbial growth, eliminating or lowering the need for preservative compounds that may irritate the skin. However, low pH lotions pose technical challenges because many of the emollients, emulsifiers, rheology additives, and other desirable components of a lotion composition are not stable or do not function well at low pH.

Some lotion compositions are used to partially or completely load a nonwoven substrate. It may be desirable for the substrate to be loaded with lotion to a degree that some of the lotion can be easily transferred to the skin during use. This transfer may facilitate cleaning, provide pleasant sensations for the user (such as smooth skin feeling or coolness from evaporation), and allow for the transfer of compounds to provide beneficial functions on the skin. These compounds may include film formers, emulsifiers, and surfactants that may make clean-up easier, or compounds to soothe, soften, or otherwise improve the condition of the skin or its appearance.

Nonwoven substrates may have a high density of interstitial spaces between the fibers making up the substrate. In order to maintain enough lotion available on the surface of a wipe to transfer to the skin, much of the interstitial space in a wipe may be filled with lotion. Because a substantial portion of this “interstitial liquid” may be isolated away from the surface of the wipe, it might not be readily available to facilitate cleansing or for transfer to the skin. The interstitial lotion, which may be the majority of the lotion associated with a wipe, therefore might not function to provide benefits associated with cleaning and caring for the skin. In order to make more lotion available for transfer to the skin, excess lotion can be loaded into the wipe, and this amount may be significant in order to provide an adequate sense of wetness, both in terms of sensory benefits (such as cooling and smoothness sensations) and as a signal that the wipe is cleaning well during use. But, some of the excess lotion may be a waste of the composition, and, therefore, adds an unnecessary cost. In addition, the excess lotion may add unnecessary weight to the wipe, thereby reducing transportation efficiencies and negatively influencing the environmental impact of the distribution process.

Accordingly, it would be desirable to provide a low pH lotion composition providing skin care benefits with an optimum lotion load to improve the amount of the lotion that is providing a meaningful function to the end-user. It would also be desirable to provide a lotion composition which can provide signals of wetness with reduced lotion loading.

### SUMMARY OF THE INVENTION

A wet wipe may comprise an aqueous lotion composition releasably carried by a substrate. The lotion composition may comprise a water soluble superwetter and a shear-thinning rheology modifier. The superwetter may have an average molecular weight of less than about 3,000, or less than about 1,500. The superwetter may be PEG-8 dimethicone. The superwetter may be present at 0.01% to 0.2% weight of the superwetter to weight of the lotion composition. The rheology modifier may be present at 0.01% to 0.5% weight of the rheology modifier to weight of the lotion composition.

The lotion composition may further comprise an emollient and an emulsifier. The lotion composition may have a pH equal to or more acidic than 4.5. The emollient, if present, may be a thickening emollient. The emulsifier, if present, may be solid at room temperature. The wet wipe may further comprise a preservative. The lotion load may be between 150% and 480%. For a fixed substrate and wetness sensory rating the ratio of the lotion load of the lotion of Claim 1 to the lotion load of the lotion of Example 1 may be 0.6 to 0.9. The superwetter may be selected from the group consisting of trisiloxanes; polyether dimethicones wherein the polyether functionality is PEG, PPG, or mixtures thereof; and mixtures thereof. The rheology modifier may be selected from the group consisting of xanthan gum, modified xanthan gums, and mixtures thereof.

The emollient, if present, may be selected from the group consisting of PEG-10 sunflower oil glycerides; sunflower oil, palm oil, olive oil, emu oil, babassu oil, evening primrose oil, palm kernel oil, cod liver oil, cottonseed oil, jojoba oil, meadowfoam seed oil, sweet almond oil, canola oil, soybean oil, avocado oil, safflower oil, coconut oil, sesame oil, rice bran oil, grape seed oil; mineral oil; isopropyl stearate, isostearyl isononanoate, diethylhexyl fumarate, diisostearyl malate, triisocetyl citrate, stearyl stearate, methyl palmitate, methylheptyl isostearate; petrolatum; lanolin oil and lanolin wax; long chain alcohols like cetyl alcohol, stearyl alcohol, behenyl alcohol, isostearyl alcohol, 2-hexyldecanol; myristyl alcohol; dimethicone fluids of various molecular weights and mixtures thereof; PPG-15 stearyl ether (also known as arlatone E); shea butter; olive butter; sunflower butter; coconut butter; jojoba butter; cocoa butter; squalane and squalene; isoparaffins; polyethylene glycols of various molecular weights; polypropylene glycols of various molecular weights; and mixtures thereof.

The emulsifier, if present, may be selected from the group consisting of laureth-23; ceteth-2; ceteth-10; ceteth-20; ceteth-21; ceteareth-20; steareth-2; steareth-10; steareth-20; oleth-2; oleth-10; oleth-20; steareth-100; steareth-21; PEG-40 sorbitan peroleate; PEG-8 stearate; PEG-40 stearate; PEG-50 stearate; PEG-100 stearate; sorbitan laurate; sorbitan palmitate; sorbitan stearate; sorbitan tristearate; sorbitan oleate; sorbitan trioleate; polysorbate 20; polysorbate 21; polysorbate 40; polysorbate 60; polysorbate 61; polysorbate 65; polysorbate 80; polysorbate 81; polysorbate 85; PEG-40 hydrogenated castor oil (also known as Emulsogen HCW-049); and citric acid ester (also known as Citrem N12 Veg K). The emollient, if present, may be selected from the group consisting of microcrystalline wax; paraffin wax; beeswax; carnauba wax; ozokerite wax; cetyl alcohol; stearyl alcohol; cetearyl alcohol; myristyl alcohol; behenyl alcohol; and mixtures thereof.

A method of increasing the perception of wetness in a wet wipe may comprise providing a lotion, the lotion comprising a water soluble superwetter and a rheology modifier; providing a wipe substrate; and loading the wipe substrate with the lotion.

A wet wipe may have a wetness sensory rating to lotion load ratio of at least 4.5.

### BRIEF DESCRIPTION OF THE DRAWINGS

FIG. 1 is a microscopic view of an exemplary nonwoven substrate.

FIG. 2 is a graph of Sensory Wetness Rating plotted against Molecular Weight of PEG-8 dimethicone component for exemplary lotion compositions according to the description below.

FIG. 3 is a graph of Sensory Wetness Rating plotted against Lotion Load for an exemplary lotion according to the description below and control lotion compositions.

FIG. 4 is a graph of Sensory Wetness Rating plotted against Lotion Load for an exemplary lotion according to the description below on various substrates.

FIG. 5 is a graph of Sensory Wetness Rating plotted against Xanthan Gum concentration for exemplary lotion compositions according to the description below.

FIG. 6 is a graph of Artificial Bowel Movement (ABM) Pick-up (ABM picked up by wipe as percent of a known quantity of ABM applied to test surface) plotted against lotion load and type for an exemplary lotion according to the description below and a control lotion.

FIG. 7 is a graph of Sensory Wetness Rating plotted against Lotion Load for various marketed wipes and an exemplary wipe according to the description below.

### DETAILED DESCRIPTION OF THE INVENTION

“Load,” as used herein, refers to combining a wipe substrate with a lotion composition, i.e., a lotion composition is loaded onto or into a wipe substrate, without regard to the method used to combine the wipe substrate with the lotion composition, i.e., immersion, spraying, kissrolling, etc. A “lotion load” refers to the amount of lotion loaded onto or into a wipe or substrate, and is expressed as weight of the lotion to weight of the dry (unloaded) wipe substrate, as a percentage.

“Molecular weight,” as used herein, refers to the weight average molecular weight (M_{w}).

“Water solubility,” as used herein, refers to the maximum amount of a substance that can dissolve in water at equilibrium at a given temperature and pressure. For the purposes of the present disclosure, a compound is considered water soluble if about 1% w/w or greater of 100% active material completely dissolves in water at 25° C. and approximately 1 atm.

The percent-weight ranges provided in the examples and discussion assume all materials are approximately 100% active, unless otherwise stated. It should be understood that the ranges may require adjustment for materials at lower active concentrations.

The present disclosure relates to a lotion composition for wet wipes. Wet wipes or wipes or wet-tissues are general terms to describe a wetted piece of material, such as non-woven material, used to cleanse body parts. Some currently available wipes are intended for the cleaning of the perianal area after defecation or urination. Other wipes are available for the cleansing of the face, or other body parts, or inanimate objects, like countertops. Wet wipes are generally of sufficient dimensions to allow for convenient handling while being small enough to be easily disposed of in the sewage system or in household garbage bins. The material of the wipes may be soft and flexible, and may have a textured surface to enhance cleaning performance and/or provide a pleasant feel to the skin. The material may be a non-woven material, and may be made of synthetic compounds. However, woven materials as well as the use of natural compounds like cotton or cellulose in either woven or nonwoven materials are within the scope of the present disclosure. In addition, synthetic and natural compounds can be combined to make up the woven or nonwoven materials. The texture and material of the wipe may affect the cleaning performance of the wipe. Some exemplary non-woven materials comprise fibers selected from the groups consisting of polyolefins, polyesters, celluloses, rayons, polyamides, polyesteramides, polyvinyl alcohols, and combinations thereof. A substrate may be manufactured via any process, such as, but not limited to, spunlace, meltblowing, airlaying, coform, and carding processes, and may have a dry basis weight of between about 25 grams per square meter (gsm) and 75 gsm, or between 30 gsm and 65 gsm. Other substrates may be used with the lotion compositions described below.

Wet wipes may be impregnated with a liquid or semi-liquid composition, the composition being referred to as a lotion. The lotion may enhance the cleaning of a surface such as skin and/or provide a smooth feeling on the skin during and after using the wet wipe. The viscosity of the composition may be balanced to homogenously impregnate the structure of the wipe (favoring a low viscosity) and to prevent the lotion composition from settling to the base of the tub during shipping, storage, or use (favoring a high viscosity). In some instances, the liquid composition may be primarily present at the wipe surface and to a lesser extent in the inner structure of the wipe. In some embodiments, the composition is releasably carried by the material, that is, the composition is contained either in or on a substrate and is readily releasable from the substrate by applying some force to the substrate by, for example, wringing the substrate or wiping a surface, such as a child's bottom, with the wet wipe.

### Wetness

It may be desirable for a wet wipe to feel wet. Humans do not have “wet” receptors in the same way that we have receptors physiologically adapted to sense heat, for example, or mechanical deformation. Instead, the sensation of wetness appears to be an integration of many different perceptions. Some key features of wetness are believed to be liquid availability (something present in sufficient quantity to be felt or perceived), evaporative cooling, the rate and extent of spreading of liquid over the skin, a perception of slipperiness, and visual aspects (such as perceived sheen or glossiness).

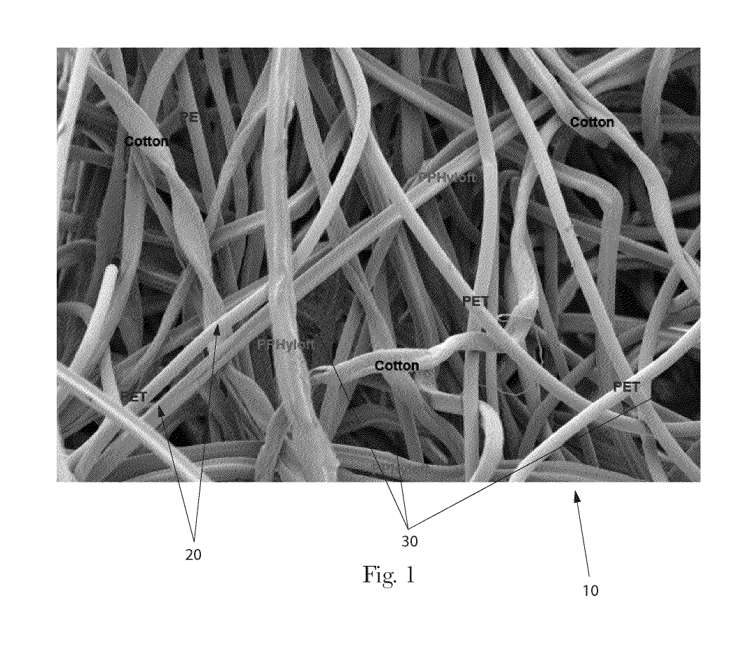

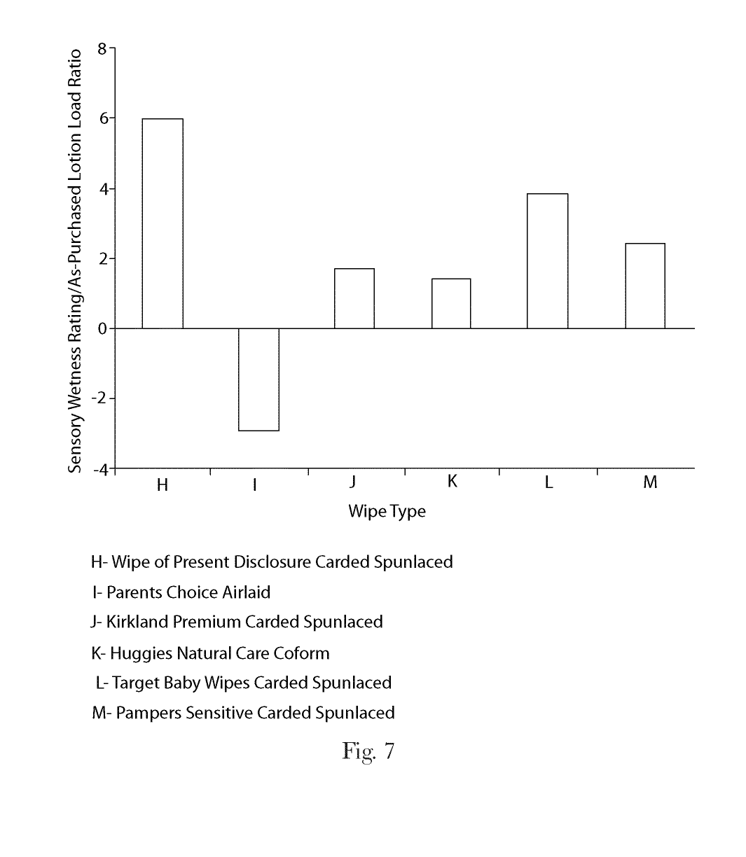

FIG. 1 shows a top view, magnified approximately 20 times, of an exemplary, carded wipe substrate 10 having fibers 20 and interstices 30. Wetness in a wet wipe may be conveyed by loading the wipe to a degree that sufficient liquid is available on the surface of the wipe for transfer to the skin. However, this requires excess lotion loading to ensure transfer of a sufficient amount of liquid to fill a substantial portion of the interstitial space in the wipe. It has been discovered that an equivalent perception of wetness can be maintained using less liquid by formulating a lotion composition with superior spreading and slipperiness characteristics. Such a lotion may feel as wet as traditional lotions, but at a significantly lower volume of transferred liquid. This can be seen in FIG. 7, which shows the ratio of wetness sensory rating to the lotion load for several commercially available wet wipes and a lab-made wet wipe with a lotion according to the present disclosure. The wetness sensory rating is obtained using the test method described below.

The rate and extent of lotion spreading can be enhanced by adding or increasing the concentration of a super wetting agent or superwetter. Similar to a typical surfactant, a superwetter is a compound that reduces surface tension. However, without being bound by theory, it is thought that, beyond reducing the overall surface tension of the liquid, a superwetter preferentially migrates to the leading edge of a liquid on a surface, creating a self-perpetuating gradient of surface tension that further drives the spreading of the liquid over the surface. When touched, a liquid composition comprising a superwetter may spread more easily and/or quickly over the skin, creating the impression of a higher volume of liquid. The greater spread may also increase the rate and/or extent of evaporative cooling, further reinforcing the perception of a higher volume of liquid. Collectively, the improved spreading and evaporative cooling may be perceived as increased wetness by a person using the wipe.

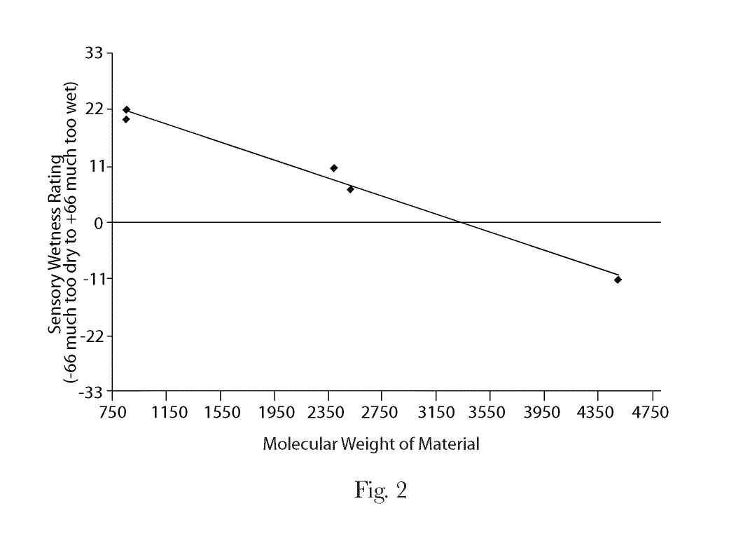

A superwetter can be distinguished from a surfactant using the Standard Test Method for Evaluation of Wetting Agents, as described in AATCC Test Method 17-2005. A superwetter may have a time for full wetting less than 30 seconds at 0.1% concentration (weight of superwetter to weight of distilled water) and 25° C. Examples of superwetters useful in a lotion composition for a wet wipe may include trisiloxanes; polyether dimethicones having a polyether functionality of PEG, PPG, or mixtures of PEG and PPG; and mixtures thereof. Specific, non-limiting, examples of polyether dimethicones include Silsurf A208, Silsurf B208, and Silsurf C208, available from Siltech; Silsoft 810, Silsoft 840, Silsoft 850, and Silwet L-7607, available from Momentive Performance Materials; Q2-5211 from Dow Corning; Zenicone IX from Zenitech; and Fibrosil SC-C8 from Innovative Specialties. Several of these are tradenames for PEG-8 Dimethicone products. However, not all materials with an INCI designation of PEG-8 Dimethicone are equivalent. PEG-8 Dimethicone may have a molecular weight range of approximately 800 to 4,000 or even 5,000. Typically, a given PEG-8 Dimethicone product will have a distribution of molecular weights, with different products from different suppliers having different averages, although the average for a single product from a single manufacturer may be consistent over time. The lower molecular weight products, less than or equal to approximately 1,500, appear to provide superior superwetting, with some degradation in performance as the molecular weight approaches 2,500, and further degradation in performance as the molecular weight exceeds 3,000. FIG. 2 is a graph showing the sensory wetness rating for a lotion formulated with a superwetter of different molecular weights, with a regression line showing the general trend of decreasing sensory wetness rating as the molecular weight of the superwetter increases. Sensory wetness ratings are obtained using the test method described below.

Other superwetters include trisiloxanes, such as Silsoft TT, available from Momentive Performance Materials. Low molecular weight molecules, that is, molecules having a molecular weight less than 2,500, or even less than 1,500, again tend to provide better superwetting performance. If a trisiloxane is used as the superwetter for a low-pH lotion, care should be taken to select a compound that is stable at the pH of the lotion formula to ensure that the superwetter will continue to function in a lotion composition over time.

Silicone-based compounds and functionalized silicone-based compounds may be used as emulsifiers or emollients. It will be appreciated that emulsifiers and/or emollients will be of a much higher molecular weight than 3,000, so that they can stabilize other materials within their structure and/or provide skin care benefits associated with emollients. Higher molecular weight emollients may be associated with substantivity of the composition on the skin surface, that is, high molecular weight emollients may be more noticeable to the touch, and may reside on the skin surface for a longer time. Emollient skin care benefits may include providing a soft appearance of the skin, lubricating the skin to reduce friction, and increasing the water content of the skin to improve moisturization and reduce skin flaking by occluding the skin to reduce water evaporation. The selection of specific compounds for use as a superwetter follows different criteria than the selection of emulsifiers and emollients, even within the same chemical genus. For example, ABILCARE-85 is an ingredient sometimes found in lotion compositions. ABILCARE-85 may function as both an emulsifier and emollient. However, ABILCARE-85 and similar higher molecular weight compounds do not function as superwetters, and they can actually impede the function of a superwetter when combined with a superwetter. High molecular weight emulsifiers may emulsify and inactivate the superwetter, especially if they both contain similar chemical moieties. If the superwetter is complexed with a higher molecular weight emulsifier, it cannot migrate to the leading edge of the liquid droplet, and therefore cannot facilitate superior liquid spreading, as measured by the Standard Test Method for Evaluation of Wetting Agents, as described in AATCC Test Method 17-2005.

Further, some emulsifiers and emollients may be only partially water soluble, in order to facilitate emulsification of lipophilic components like emollients within an oil-in-water lotion composition. The superwetter should be water soluble in aqueous formulations or in oil-in-water formulations. A compound which is not inherently miscible in water will not be able to migrate to the leading edge of an aqueous composition, and, therefore, would not function as a superwetter.

The superwetter may be added to a lotion composition at a level ranging from 0.01% to 0.3% or more weight of the superwetter to weight of the lotion composition. In some embodiments, a superwetter concentration of 0.1% w/w may be the maximum concentration at which increasing benefit is obtained from adding additional superwetter to the composition. The lotion composition may have an overall, static surface tension of less than 40 mN/m.

Lotion slip can be enhanced through the selection of a rheology modifier. Rheology modifiers are compounds that may increase the viscosity of the composition at both lower temperatures as well as at potentially higher process, storage, or transportation temperatures. Rheology modifiers, also called suspending agents, stabilizers, or viscosity control agents, provide “structure” to the compositions to reduce the settling out (separation) of insoluble and partially soluble components. Rheology modifiers may also aid in reducing the settling of the lotion to the bottom of the packaging material during transportation, storage, and use. Rheology modifiers may also aid in stabilizing an emulsion, by reducing the probability of phase separations in the lotion.

The manner in which rheology modifiers build viscosity through creating structure in the composition differs based on their chemistry. For compositions containing shear thinning rheology modifiers, the introduction of shear and pressure, such as from moving a wipe across the skin, causes the interactions between molecules of the rheology modifier to weaken and break down, resulting in a significant drop in viscosity once sufficient shear and pressure has been applied. This breakdown of bonds between molecules of the rheology modifier may generate a sensation of slip. Without wishing to be bound by theory, shear thinning rheology modifiers that form a gel-like crystal structure may be best suited for generating slip because parts of the crystal structure are able to glide along each other when shear is applied.

A lotion composition may have a viscosity at low shear of greater than about 4,000 centipoise, or greater than about 7,000 centipoise, or even greater than about 10,000 centipoise at a shear stress of about 0.05 Pascal (Pa) when measured using the shear stress profile using a plate and cone rheometer according to the method described below. The chemistry, amount and/or molecular weight of the rheology modifier may be selected to provide this viscosity range in the lotion composition.

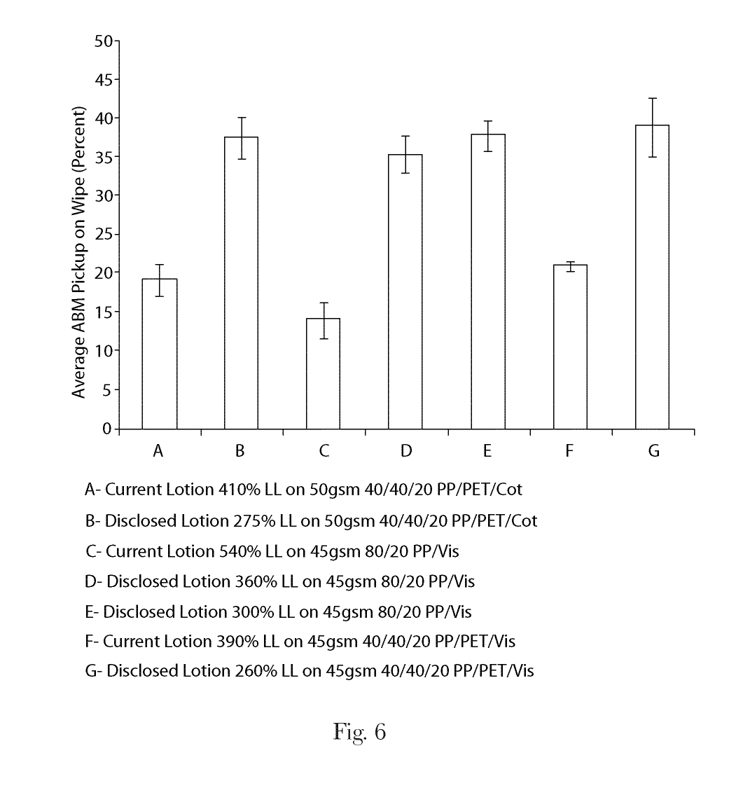

In some wipes (such as those described in the art with compositions comprising silicone-based compounds or functionalized silicone-based compounds), increasing the slip of the lotion might result in the wipe also having increased slip. Too much slipperiness of the wipe surface may be undesirable because it can reduce the interaction of the wipe with the soil being cleaned, resulting in smearing of the soil and/or poor soil capture on the wipe. Increased slipperiness of the wipe may also make it difficult for the person using the wipe to control the cleaning process, as the wipe may tend to slide off the hand and to fold upon itself In wipes of the present disclosure, the slip of the lotion may be increased without increasing the slip of the wipe. By combining a rheology modifier with a super wetting agent, the amount of liquid required to achieve the desired sensation of wetness may be reduced. Loading the wipe with less liquid may have two relevant effects. First, there may be less fluid present at the interface of the wipe and soil, resulting in greater mechanical interaction between the fibers of the substrate and the soil being cleaned. Second, there may be greater void space within the wipe available to capture the soil because less of the interstitial space is occupied with liquid. The overall result may be a wipe that feels suitably wet but has good adhesion between the wipe surface and the soil being cleaned. As shown in FIG. 6, a lotion according to the present disclosure may be associated with improved pick-up of Artificial Bowel Movement (ABM) relative to a currently marketed lotion, and this improvement may be observed on a variety of substrate types. Where observed, this improved adhesion or pick-up of ABM may correlate to faster or easier clean-up of real fecal material or other body exudates.

Exemplary rheology modifiers for improved slip include, but are not limited to, sodium polyacrylate; ammonium acrylodimethyltaurate/VP copolymer; a mixture of caprylic/capric triglyceride and ammonium acryloyldimethyltaurate/VP copolymer, trilaureth-4 phosphate and polyglyceryl-2 sesquiisostearate, available as Aristoflex AVL from Clariant; a mixture of caprylic/capric triglyceride and ammonium acryloyldimethyltaurate/beheneth-25 methacrylate crosspolymer, available as Aristoflex HML from Clariant; ammonium acryloyldimethyltaurate/beheneth-25 methacrylate crosspolymer, available as Aristoflex HMB from Clariant; a mixture of sodium polyacrylate, C13-14 isoparaffin, and trideceth-6, available as Aristoflex PAL 30 from Clariant; a mixture of sodium polyacrylate, hydrogenated polydecene, and trideceth-6, available as Aristoflex PAL 57 from Clariant; acrylic acid/VP crosspolymer, available as Ultrathix P-100 from ISP; acrylates/C10-30 alkyl acrylate crosspolymer, available as CARBOPOL Ultrez 20, from Lubrizol; a mixture of sodium acrylate/sodium acryloyldimethyl taurate copolymer, isohexadecane, and polysorbate 80, available as Simulgel EG from Seppic; a mixture of hydroxyethyl acrylate/sodium acryloyldimethyl taurate copolymer, isohexadecane, and polysorbate 60, available as Simulgel INS 100 from Seppic; a mixture of polyacrylate-X, isohexadecane, and polysorbate 60, available as Simulgel SMS 88 from Seppic; a mixture of polyacrylamide, C13-14 isoparaffin, and laureth-7, available as Sepigel 305 from Seppic; acrylates/vinyl isodecanoate crosspolymer available as Stabylen 30 from 3V; acrylates/C10-30 alkyl acrylate crosspolymer, available as Pemulen TR1 and TR2 from Lubrizol; hydrocolloids of plant or biosynthetic origin, for example, xanthan gum, karaya gum, alginates, sclerotium gum, galactoarabinan, diutan gum, guar gum, locust bean gum, and gellan gum; fumed silicas and treated silicas; silicates; starch and its hydrophilic derivatives; polyurethanes; and mixtures thereof.

Exemplary rheology modifiers for improved slip in low pH formulations include, but are not limited to, dehydroxanthan gum, such as Amaze XT, available from Akzo Nobel; xanthan gum, such as Keltrol SFT or Keltrol CGSFT, available from CP Kelco, Xanthan FG or Xanthan FN, available from Jungbunzlauer; and mixtures thereof.

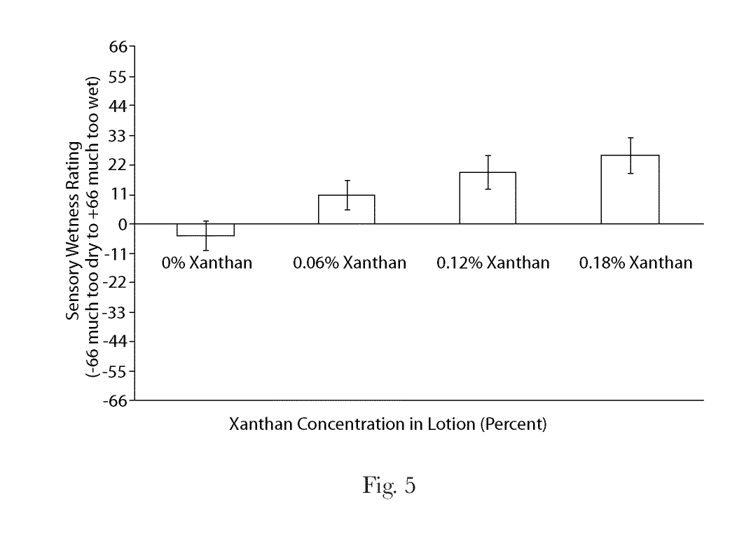

The rheology modifier may be present in a lotion composition at a level of 0.01% to 0.5% weight of the rheology modifier to weight of the lotion composition. Depending upon the rheology modifier used and the other components of the lotion composition, higher levels of a rheology modifier may create an undesirable sensation of stickiness or tackiness rather than increased slip. FIG. 5 shows the effect of modifying only the rheology modifier concentration on the wetness sensory rating of a lotion according to the present disclosure, with increasing wetness sensory ratings for a range of rheology modifier from 0.06% to 0.18%.

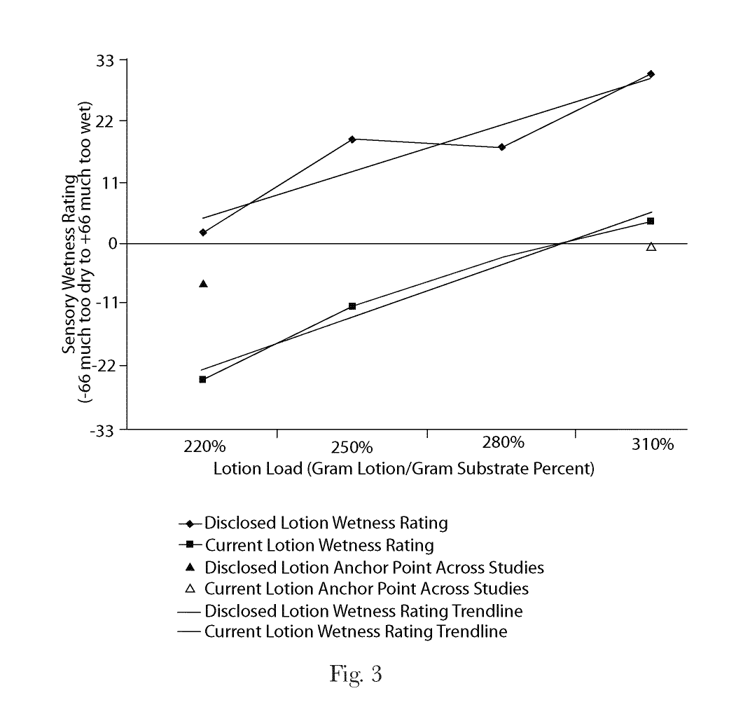

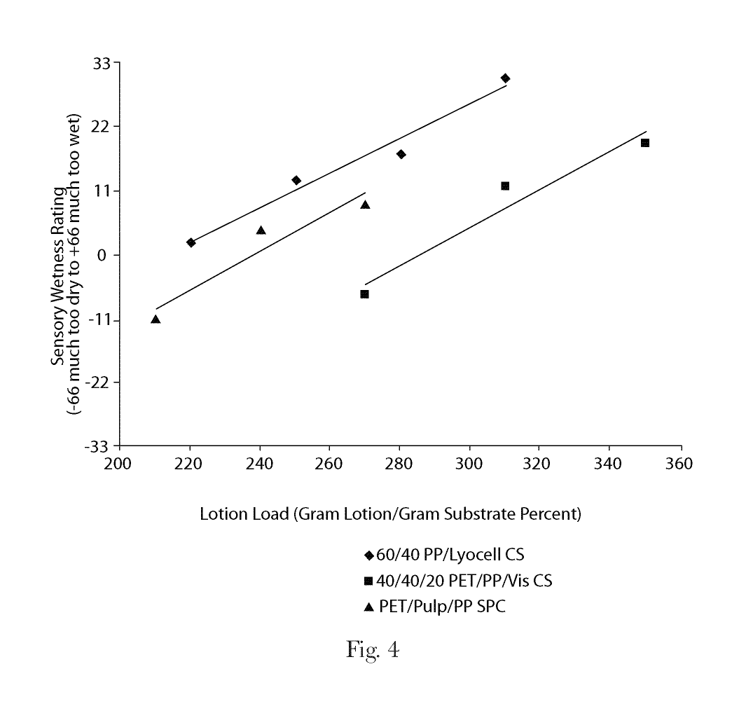

Using a combination of a superwetter and a rheology modifier, it may be possible to reduce the lotion load on a wipe by ⅛, or ¼, or ⅓, or even ½, without detrimentally changing the perception of the wetness of the wipe. As an example, for a carded spunlaced wipe of 60%/40% polypropylene/viscose with a basis weight of 58 gsm and a lotion load of 315%, a composition containing a combination of a superwetter and a rheology modifier enables this load to be reduced to 200% and still achieve parity sensory wetness. This improvement can be achieved without altering the structure of the wipe substrate (e.g., to reduce the interstitial space in the wipe) or changing the process for loading the wipe substrate with the lotion composition. FIG. 3 shows that the wetness sensory rating of a lotion according to the present disclosure is consistently higher than the wetness sensory rating of a currently marketed wipe lotion across a range of lotion loads on a specific substrate. As shown in FIG. 4, a lotion according to the present disclosure can maintain or improve wetness sensory ratings at lower lotion loads on substrates of various fiber types and formation processes. For any individual wipe, the reduction in lotion load may be small in absolute value. However, cumulatively over thousands of packages, the savings in conserved materials and lower transportation weight may be substantial.

In some embodiments, for a given substrate, the ratio of the lotion load of a lotion as taught herein required to achieve a given wetness rating to the lotion load of a control lotion (such as the lotion of Example 1) required to achieve the same wetness rating may be 0.6 to 0.9. The lotion load using a lotion as taught herein may range, for example, from 150% to 480%. A wet wipe as taught herein may have a wetness sensory rating to lotion load ratio greater than 4.5, for example, or greater than 5, or between 4.5 and 6, or between 4.5 and 7.

Any of the compositions described above may be used to increase the perception of wetness in a wet wipe. A method for increasing the perception of wetness in a wet wipe may include providing a lotion composition as described above, providing a wipe substrate, and loading the wipe substrate with the lotion.

### Skin Care

Many emollients might desirably be included in a lotion composition, however, a lotion composition often includes components with competing or incompatible properties, and therefore it is not always possible to incorporate a given emollient into a new lotion. For example, sunflower oil is an emollient which has desirable skin care effects, such as soothing, softening, and caring for the skin, and is naturally occurring, which may appeal to users who are concerned about the health effects of long-term exposure to synthetic compounds. However, sunflower oil is lipophilic and immiscible in water, and is therefore difficult to incorporate into a water-based lotion. Further, sunflower oil, like many emollients and emulsifiers, is subject to potential hydrolysis in a low pH composition, and is therefore difficult to incorporate into and stabilize in a low pH composition.

It has been found that improved stability of some emollients and emulsifiers in a low pH, aqueous composition can be achieved by providing increased structure at the water/lipid interface in the composition. Emulsifiers that are solids at room temperature provide a more rigid and durable micellar structure around the oil droplet such that coalescence between the droplets is reduced, and, therefore, the system is more stable and less prone to separation, hydrolysis, and other common problems with lotion compositions. This more stable micellar structure surrounding the oil droplets at the water/lipid interface can be increased by not only using an emulsifier which is solid at room temperature, but also by incorporating materials like fatty alcohols. Solid emulsifiers and fatty alcohols may also generate a desirable “silky” feel after the lotion composition has dried on the skin, whereas emulsifiers which are liquid at room temperature may leave a oilier and greasy feeling after the water from the lotion composition evaporates.

The stability of oil-in-water emulsions made with lipophilic emollients like sunflower oil can be further improved by using a rheology modifier to increase the static viscosity of the water continuous phase of the lotion composition. Increasing the static viscosity of the water continuous phase of the lotion composition reduces the probability that the oil droplets will collide and coalesce within the lotion composition. This coalescence could lead to phase separation and, ultimately, an unstable lotion composition. Ideally, a rheology modifier might be present from 0.01% to 0.3% weight of the rheology modifier to weight of the lotion composition. However, as described above, high levels of some rheology modifiers may lead to a sticky or tacky feel. Accordingly, it may be desirable to use a lower level of a rheology modifier to reduce undesirable tactile effects and save on costly raw materials. It has been found that a stable, aqueous lotion composition having a low pH and increased spreading and slip characteristics can be formulated using rheology modifier levels of 0.05% to 0.3% weight of the rheology modifier to weight of the lotion composition when the rheology modifier is Xanthan Gum.

Exemplary emollients for use in aqueous lotion compositions having a low pH and increased spreading and slip characteristics include, but are not limited to, PEG-10 sunflower oil glycerides; vegetable oils like sunflower oil, palm oil, olive oil, emu oil, babassu oil, evening primrose oil, palm kernel oil, cottonseed oil, jojoba oil, meadowfoam seed oil, sweet almond oil, canola oil, soybean oil, avocado oil, safflower oil, coconut oil, sesame oil, rice bran oil, and grape seed oil; mineral oil; esters like isopropyl stearate, isostearyl isononanoate, diethylhexyl fumarate, diisostearyl malate, triisocetyl citrate, stearyl stearate, methyl palmitate, and methylheptyl isostearate; petrolatum; lanolin oil and lanolin wax; long chain alcohols like cetyl alcohol, stearyl alcohol, behenyl alcohol, isostearyl alcohol, 2-hexyldecanol and myristyl alcohol; dimethicone fluids of various molecular weights and mixtures thereof; PPG-15 stearyl ether (also known as arlatone E); shea butter; olive butter; sunflower butter; coconut butter; jojoba butter; cocoa butter; squalane and squalene; isoparaffins; polyethylene glycols of various molecular weights; polypropylene glycols of various molecular weights; and mixtures thereof.

Some emollients may also act as thickeners (viscosity-increasing agents, although perhaps not rheology modifiers in the sense of structuring the composition). Such thickening emollients include, but are not limited to, hydrogenated vegetable oils like hydrogenated jojoba oil and hydrogenated jojoba wax; microcrystalline wax; paraffin wax; beeswax; carnauba wax; ozokerite wax; ceresine wax; myristyl alcohol; behenyl alcohol; cetyl alcohol; stearyl alcohol; cetearyl alcohol; and mixtures thereof.

Exemplary rheology modifiers for a stable, aqueous lotion composition having a low pH and increased slip characteristics include, but are not limited to, those described above.

Emulsifiers may be desirable to help incorporate otherwise immiscible emollients into a water-based lotion composition. As discussed above, very high molecular weight emulsifiers, especially those with moieties related to those in the superwetter, may interfere with the performance of the superwetter, if present. Exemplary emulsifiers for use in a stable, aqueous lotion composition having a low pH and increased spread and slip characteristics include, but are not limited to, ethoxylated alcohols like laureth-23, ceteth-2, ceteth-10, ceteth-20, ceteth-21, ceteareth-20, steareth-2, steareth-10, steareth-20, steareth-21, oleth-2, oleth-10, oleth-20, steareth-100, steareth-21; ethoxylated alkylates like PEG-8 stearate, PEG-40 stearate, PEG-2 stearate, PEG-50 stearate, PEG-20 palmitate, PEG-2 palmitate, and PEG-100 stearate; sorbitan monoalkylates like sorbitan stearate; sorbitan laurate; sorbitan oleate, and sorbitan palmitate; other alkylated sorbitans like sorbitan tristearate, sorbitan sesquioleate, and sorbitan trioleate; ethoxylated sorbitans like polysorbate 20, polysorbate 21, polysorbate 40, polysorbate 60, polysorbate 61, polysorbate 65, polysorbate 80, polysorbate 81, PEG-40 sorbitan peroleate, and polysorbate 85; PEG-40 hydrogenated castor oil (also known as Emulsogen HCW-049); citric acid esters (such as Citrem N12 Veg K from Danisco Inc.); lactic acid esters; acetic acid esters; alkyl polyglycosides; sulfosuccinates and sulfosuccinate derivatives such as sodium dioctyl sulfosuccinate; and mixtures thereof.

### Optional Components

A lotion composition may optionally include adjunct ingredients. Possible adjunct ingredients may be selected from a wide range of additional ingredients including, but not limited to perfumes and fragrances, texturizers, anti-oxidants, pH buffers, metal sequestrants, colorants, and medically active ingredients, in particular healing actives, humectants, anti-stick agents, and skin protectants. Other adjuncts include preservatives. A low pH lotion composition, although hostile to many microorganisms, may include preservatives to protect against acidophiles or to extend the shelf life of the lotion composition. Exemplary preservatives for a low pH lotion composition include, but are not limited to, citric acid-sodium citrate buffer, benzyl alcohol, sodium benzoate, mixtures of phenoxyethanol and ethylhexylglycerin, and mixtures thereof.

Acids, bases, and pH buffers may be used to adjust the pH of the lotion composition as desired. For example, it may be desirable to adjust the lotion composition to a pH no higher than 5.5 or 6, because the natural pH of clean, healthy skin is approximately 5. It may be desirable to further adjust the pH of the lotion composition below 5, or below 4, to compensate for alkaline exudate residues that may remain on the skin after cleaning with a wet wipe and to render the composition less hospitable to microorganisms. A pH much below 3.5 may be undesirable as it may cause stinging or irritation of the skin, particularly when a wipe is used in the absence of significant alkaline residues. A pH between 3.5 and 4.5, for example, may be desirable. Exemplary compounds for adjusting the pH of a lotion composition include, but are not limited to, citric acid, trisodium citrate, sodium phosphate, gluconic acid, glycolic acid, sodium gluconate, sodium glycolate, malic acid, lactic acid, sodium lactate, sodium hydroxide, sodium malate, other fruit acids and their conjugate bases, and mixtures thereof.

A lotion composition may comprise skin-care ingredients, including antioxidants such as vitamin E, citric acid, ascorbic acid, rosemary, and/or BHT. A lotion composition may further comprise particulates that deliver a desirable smooth, silky feel to the skin, such as Tospearl, available from Momentive; talc; mica; nylon particles; polyethylene particles; silica particles; and starch.

### EXAMPLES

Example 1 provides a control lotion composition, not formulated according to the present disclosure.

Example 1 (Control Lotion)

| Components | Weight Percent |
|---|---|
| Water | Q.S. |
| Disodium EDTA | 0.100 |
| Xanthan Gum^{†} | 0.180 |
| Sodium Benzoate | 0.120 |
| PEG-40 Hydrogenated Castor Oil | 0.440 |
| Citric Acid | 0.530 |
| Trisodium Citrate | 0.330 |
| Benzyl Alcohol | 0.300 |
| Euxyl ® PE9010 | 0.300 |
| Abil Care 85 | 0.100 |

^{†}Xanthan FG from Jungbunzlauer, Newton Center, MA

The following examples provide exemplary embodiments of lotion compositions according to the present disclosure.

| Example 2 | Trade Name | Source | Activity | Wt. % |
|---|---|---|---|---|
| Water | Dihydrogen | lab DI | 1 | Q.S. |
| | oxide | | | |
| Xanthan Gum | Xanthan Gum | Jungbunzlaer | 1 | 0.18% |
| | FG | | | |
| Sunflower Oil | NuSun | Cargill | 1 | 4.78% |
| Polyoxyeth- | Brij 72 | Croda | 1 | 1.19% |
| ylene 2 | | | | |
| Stearyl Ether | | | | |
| Polyoxyeth- | Brij 78 | Croda | 1 | 0.53% |
| ylene 20 | | | | |
| Stearyl Ether | | | | |
| Cetyl Alcohol | CO-1695 | P&G | 1 | 0.50% |
| Phenoxyethanol | Euxyl 9010 | Schulke | 1 | 0.31% |
| and Ethyl- | | | | |
| hexylglycerin | | | | |
| Benzyl Alcohol | Benzyl Alcohol | | 1 | 0.32% |
| Sodium | Sodium | | 1 | 0.12% |
| Benzoate | Benzoate | | | |
| Disodium EDTA | Disodium EDTA | | 1 | 0.10% |
| Anti-Stick | Dermal Care | Rhodia | 0.697 | 2.10% |
| Polymer | ED-307/s | | | |
| PEG-8 | Silsurf A-208 | Siltech | 1 | 0.20% |
| Dimethicone | | | | |
| Trisodium | Trisodium | | 1 | 0.16% |
| Citrate | Citrate | | | |
| Citric Acid | Citric Acid | | 1 | 0.35% |
| FINAL pH = | | | | |
| 3.75 | | | | |
| Total | | | | 100.00% |

| Example 3 | Trade Name | Source | Activity | Wt. % |
|---|---|---|---|---|
| Water | Dihydrogen | lab DI | 1 | Q.S. |
| | oxide | | | |
| Xanthan Gum | Xanthan Gum | Jungbunzlaer | 1 | 0.18% |
| | FG | | | |
| Sunflower Oil | NuSun | Cargill | 1 | 2.90% |
| Sorbitan | Span 60 | Croda | 1 | 0.63% |
| Monostearate | | | | |
| Polyoxyeth- | Brij 721 | Croda | 1 | 0.31% |
| ylene 21 | | | | |
| Stearyl Ether | | | | |
| Phenoxyethanol | Euxyl 9010 | Schulke | 1 | 0.30% |
| and Ethylhexyl- | | | | |
| glycerin | | | | |
| Benzyl Alcohol | Benzyl Alcohol | | 1 | 0.31% |
| Sodium | Sodium | | 1 | 0.12% |
| Benzoate | Benzoate | | | |
| Disodium EDTA | Disodium EDTA | | 1 | 0.10% |
| Anti-Stick | Dermal Care | Rhodia | 0.697 | 2.02% |
| Polymer | ED-307/s | | | |
| PEG-8 | Silsurf A-208 | Siltech | 1 | 0.20% |
| Dimethicone | | | | |
| Trisodium | Trisodium | | 1 | 0.19% |
| Citrate | Citrate | | | |
| Citric Acid | Citric Acid | | 1 | 0.44% |
| FINAL pH = | | | | |
| 3.81 | | | | |
| Total | | | | 100.00% |

| Example 4 | Trade Name | Source | Activity | Wt. % |
|---|---|---|---|---|
| Water | Dihydrogen | lab DI | 1 | Q.S. |
| | oxide | | | |
| Xanthan Gum | Xanthan Gum | Jungbunzlaer | 1 | 0.12% |
| | FG | | | |
| Sunflower Oil | NuSun | Cargill | 1 | 5.50% |
| Alkylated | Pemulen TR1 | Lubrizol | 1 | 0.10% |
| Polyacrylic | | | | |
| Acid | | | | |
| PEG-40 | Emulsogen | Clariant | 0.9 | 0.09% |
| Hydrogenated | HCW-049 | | | |
| Castor Oil | | | | |
| Premix 13 | Premix 13 | | 1 | 0.49% |
| Preservative | | | | |
| Suttocide | Suttocide | | 1 | 0.15% |
| A/50% | A/50% | | | |
| Sodium | Sodium | | 0.5 | 0.02% |
| Hydroxide/50% | Hydroxide/50% | | | |
| Disodium EDTA | Disodium EDTA | | 1 | 0.05% |
| Anti-Stick | Dermal Care | Rhodia | 0.697 | 0.56% |
| Polymer | ED-307/s | | | |
| PEG-8 | Silsurf A-208 | Siltech | 1 | 0.20% |
| Dimethicone | | | | |
| FINAL pH = | | | | |
| 5.90 | | | | |
| Total | | | | 100.00% |

| | Weight Percent | | | | |
|---|---|---|---|---|---|
| | Exam- | Exam- | Exam- | Exam- | Exam- |
| Components | ple 5 | ple 6 | ple 7 | ple 8 | ple 9 |
| Water | Q.S. | Q.S. | Q.S. | Q.S. | Q.S. |
| Disodium EDTA | 0.100 | 0.100 | 0.100 | 0.100 | 0.100 |
| Xanthan Gum^{†} | 0.180 | 0.180 | 0.250 | 0.180 | 0.250 |
| Sodium Benzoate | 0.120 | 0.120 | 0.120 | 0.120 | 0.120 |
| PEG-40 Hydro- | 0.440 | 0.440 | 0.440 | 0.440 | 0.440 |
| genated Castor | | | | | |
| Oil | | | | | |
| Citric Acid | 0.530 | 0.530 | 0.530 | 0.530 | 0.530 |
| Trisodium | 0.330 | 0.330 | 0.330 | 0.330 | 0.330 |
| Citrate | | | | | |
| Benzyl Alcohol | 0.300 | 0.300 | 0.300 | 0.300 | 0.300 |
| Euxyl ® PE9010 | 0.300 | 0.300 | 0.300 | 0.300 | 0.300 |
| PEG-8 | 0.050 | 0.100 | 0.100 | 0.200 | 0.300 |
| Dimethicone* | | | | | |
| Perfume^{Δ} | 0.070 | 0.050 | 0.070 | 0.070 | 0.050 |

*Silsurf A208 from Siltech LLC, Dacula, GA
^{†}Keltrol CGSFT from CP Kelco, Altlanta, GA or Xanthan FG from Jungbunzlauer, Newton Center, MA
^{Δ}A perfume may optionally be added at a concentration that is consumer acceptable

| | | Weight Percent | | | |
|---|---|---|---|---|---|
| | Exam- | Exam- | Exam- | Exam- | Exam- |
| Components | ple 10 | ple 11 | ple 12 | ple 13 | ple 14 |
| Water | Q.S. | Q.S. | Q.S. | Q.S. | Q.S. |
| Disodium EDTA | 0.100 | 0.100 | 0.100 | 0.100 | 0.100 |
| PEG-40 Hydro- | 0.400 | 0.400 | 0.400 | 0.400 | 0.400 |
| genated Castor | | | | | |
| Oil | | | | | |
| Propylene | 1.500 | 1.500 | 1.500 | 1.500 | 1.500 |
| Glycol | | | | | |
| Phenoxyethanol | 0.800 | 0.800 | 0.800 | 0.800 | 0.800 |
| Methyl Paraben | 0.150 | 0.150 | 0.150 | 0.150 | 0.150 |
| Ethyl Paraben | 0.050 | 0.050 | 0.050 | 0.050 | 0.050 |
| Propyl Paraben | 0.050 | 0.050 | 0.050 | 0.050 | 0.050 |
| Xanthan Gum^{†} | 0.180 | 0.180 | 0.250 | 0.180 | 0.250 |
| Trilaureth-4 | 0.400 | 0.400 | 0.400 | 0.400 | 0.400 |
| Phosphate | | | | | |
| Monobasic | 0.180 | 0.180 | 0.180 | 0.180 | 0.180 |
| Sodium | | | | | |
| Phosphate | | | | | |
| Anhydrous | | | | | |
| PEG-8 | 0.050 | 0.100 | 0.100 | 0.200 | 0.300 |
| Dimethicone* | | | | | |
| Perfume^{Δ} | 0.070 | 0.050 | 0.070 | 0.070 | 0.050 |

*Silsurf A208 from Siltech LLC, Dacula, GA
^{†}Keltrol CGSFT from CP Kelco, Altlanta, GA or Xanthan FG from Jungbunzlauer, Newton Center, MA
^{Δ}A perfume may optionally be added at a concentration that is consumer acceptable

| | | Weight Percent | | | |
|---|---|---|---|---|---|
| | Exam- | Exam- | Exam- | Exam- | Exam- |
| Components | ple 15 | ple 16 | ple 17 | ple 18 | ple 19 |
| Water | Q.S. | Q.S. | Q.S. | Q.S. | Q.S. |
| Disodium EDTA | 0.100 | 0.100 | 0.100 | 0.100 | 0.100 |
| Iodopropynyl- | 0.009 | 0.009 | 0.009 | 0.009 | 0.009 |
| butylcarbamate | | | | | |
| Benzyl Alcohol | 0.500 | 0.500 | 0.500 | 0.500 | 0.500 |
| Suttocide ® A | 0.150 | 0.150 | 0.150 | 0.150 | 0.150 |
| 50% Solution | | | | | |
| Xanthan Gum^{†} | 0.180 | 0.180 | 0.250 | 0.180 | 0.250 |
| PEG-40 Hydro- | 0.550 | 0.550 | 0.550 | 0.550 | 0.550 |
| genated Castor | | | | | |
| Oil | | | | | |
| PEG-8 | 0.050 | 0.100 | 0.100 | 0.200 | 0.300 |
| Dimethicone* | | | | | |
| Citric Acid | 0.055 | 0.055 | 0.055 | 0.055 | 0.055 |
| Perfume^{Δ} | 0.070 | 0.050 | 0.070 | 0.070 | 0.050 |

*Silsurf A208 from Siltech LLC, Dacula, GA
^{†}Keltrol CGSFT from CP Kelco, Altlanta, GA or Xanthan FG from Jungbunzlauer, Newton Center, MA
^{Δ}A perfume may optionally be added at a concentration that is consumer acceptable

### Test Methods

### Wetness Sensory Panel for Determining Wetness Sensory Rating

### A. Preparation of Wipes for the Sensory Panel

Wipes are prepared in a laboratory under room conditions of 21-24° C. and 15-40% relative humidity. Wipe substrates of the desired composition and basis weight are cut into individual 180 mm×180 mm squares using a cutting board. Gloves are worn during the process to avoid transferring body oils to the substrate. Each piece of dry substrate is then weighed and the weight in grams is recorded on a spreadsheet. The target lotion load for the wipes, i.e. grams of liquid per gram of dry substrate, is set by the individual conducting the study. For example, a lotion load known to be acceptable for the lotion of Example 1 on a 60/40 PP/Lyocell 58 gsm carded spunlaced substrate is 3.15 grams lotion/gram substrate (also expressed as 315% load).

Based on the weight of the dry substrate and the target lotion load, the target lotion weight in grams for that piece of substrate is calculated using the following equation: Dry Substrate Weight (grams) x Target Lotion Load (grams/grams) =Target Lotion Weight (grams). It is considered acceptable for the Target Lotion Weight of the wipe to vary by plus or minus 5%. The Lower Limit Lotion Weight (grams) and Upper Limit Lotion Weight (grams) are calculated by the multiplying the Target Lotion Weight (grams) by 0.95 or 1.05 respectively.

To add the lotion to the dry wipe substrate and prepare the wet wipes, 2 pieces of cut wipe substrate are lined up edge-to-edge on top of one another and immersed in a tray containing approximately 250 g of lotion. Sufficient time (approximately 2 minutes) is allowed to pass such that the wipes have completely absorbed the lotion and become saturated. The two pieces of aligned, wetted substrate are lifted out of the tray and inserted between the rollers of a Laboratory Wringer/Padder Model D394 (as available from SDL Atlas USA, Rock Hill, S.C.) while the device is turned on and the rollers are rotating. The wet wipes are grabbed by gloved hand as they pass through the rollers, being careful to keep them flat and not to crumple them. The wipes are separated, each wipe is immediately weighed, and this weight is recorded as the Converted Wipe Weight (grams). Given that the lotion load of the wipe is allowed to vary by plus or minus 5%, a Lower Limit Wet Wipe Weight (grams) and Upper Limit Wet Wipe Weight (grams) are calculated using the following equations: [Dry Substrate Weight (grams)+Lower Limit Lotion Weight (grams)]=Lower Limit Wet Wipe Weight (grams), and [Dry Substrate Weight (grams)+Upper Limit Lotion Weight (grams)]=Upper Limit Wet Wipe Weight (grams). If the weight of each wet wipe is not within the range bracketed by the Lower Limit Wet Wipe Weight and Upper Limit Wet Wipe Weight, the weight of the wet wipe must be adjusted so that it does fall within this range. Lotion load may be controlled through the use of weights (supplied by SDL Atlas with the Laboratory Wringer/Padder) that are attached to the wringer arm. In this manner, more or less pressure is applied to the rollers as the wipe passes through, resulting in more or less lotion being squeezed out. Typically, once a few wipes are run through the Laboratory Wringer/Padder and weighed, the weight applied to the wringer arm can be adjusted so that all subsequent wipes are within the desired weight target range. Any wipe passed through the Wringer that has a weight that is too low (below the Lower Limit Wet Wipe Weight) may be re-immersed in the lotion as above and run through the Wringer again using a lighter weight. Any wipe passed through the Wringer that has a weight that is too high (above the Upper Limit Wet Wipe Weight) may be run through the Wringer again using a heavier weight.

Wet wipes that have a final weight within the desired target range, and hence have the desired target load, are immediately inserted into a resealable leak-proof bag designed to have low moisture loss (Model S-5856 9 inch×12 inch 3 mil leak-proof bags as available from ULINE, Waukegan, Ill.). The wipes are inserted flat on top of one another, aligned edge-to-edge, without any visible wrinkles or crumpled regions to create a stack of 20 wipes. This stack of wipes in the sealed bag is allowed to sit overnight prior to use the next day. To ensure a homogenous distribution of lotion within the stack, each stack is inverted (flipped upside down) and left in this position for at least two hours before use in the sensory panel.

### B. Evaluation of Wet Wipes by the Sensory Panel

To quantify and compare the perceived wetness of different baby wipes, a discriminatory sensory panel is used. The panel consists of 15 randomly chosen male or female caregivers of babies (newborns to three years old) who are currently and routinely changing urine and BM diapers and who are using currently marketed baby wet wipes. None of the panelists have formal training in sensory evaluation. This purpose of this panel is to generate data that are representative of the general consumer population, i.e. that would be similar to the impressions of a consumer during home usage of the wipe.

All testing done by the sensory panel is performed under room conditions of 21-24° C. and 15-40% relative humidity.

A maximum of five different wet wipe options, each within the sealed bags described above, are randomized in order and laid out in front of a panelist on the surface of a laboratory bench. The options are identified only with a code letter of A through E. The panelist washes their hands with soap (Safeguard antibacterial liquid hand soap) and water, and then dries their hands completely with paper towel (Bounty Basics), followed by air drying at room temperature for approximately 30 seconds. One wipe from the first sealed bag is removed by the panelist (bag is then resealed) and evaluated for wetness by feeling the wipe in whatever manner the panelist prefers. The wetness of the wipe is rated by the panelist using a scale of much too wet to much too dry, and this rating is converted to a numerical score as follows:

| Panelist Rating | Numerical Score |
|---|---|
| much too wet | 66 |
| a little too wet | 33 |
| just right | 0 |
| a little too dry | −33 |
| much too dry | −66 |

After evaluating and rating the first wipe, the panelist washes their hands with soap and water and dries them completely as described above. The panelist then continues evaluating and rating each of the remaining wipes in the same manner, washing their hands in between each wipe.

This is repeated for each of the 15 panelists. It is acceptable for two evaluation stations to be set up and run simultaneously, with different panelists cycling through either station. The wipe options are re-randomized for each new panelist. Panelists may only participate in one sensory panel per day to prevent memory effects.

Wetness ratings from the panelists are entered into a spreadsheet to enable calculation of the average wetness rating for each wipe option.

| | Wetness Rating For Each Wipe Option | | | | |
|---|---|---|---|---|---|
| Panelist ID | A | B | C | D | E |
| 101 | 0 | −33 | 0 | 33 | 66 |
| 102 | 0 | −33 | 0 | 0 | 33 |
| 103 | 0 | 0 | 0 | 0 | 0 |
| 104 | 0 | −33 | −33 | 33 | 33 |
| 105 | 0 | 0 | 0 | 33 | 66 |
| 106 | 0 | 0 | −33 | 0 | 33 |
| 107 | −33 | 0 | 0 | 33 | 0 |
| 108 | 0 | 0 | 0 | 0 | 33 |
| 109 | 0 | −33 | 0 | 0 | 0 |
| 110 | 0 | 0 | −33 | 33 | 0 |
| 111 | 0 | 0 | 0 | 0 | 33 |
| 112 | 0 | −66 | 0 | 0 | 66 |
| 113 | 0 | −33 | 0 | 66 | 33 |
| 114 | 0 | 0 | −33 | 0 | 0 |
| 115 | 0 | 0 | 0 | 0 | 0 |
| Average | −2 | −15 | −9 | 15 | 26 |

Generally there will be similarity in the wetness ratings between panelists for any given wipe option, e.g. a wipe that is perceived as too dry will generally have ratings associated with the “a little too dry” and “much too dry” selections on the rating scale. As this is a subjective panel, however, there may be occasions when individual panelists provide a rating that is not consistent with the majority of other ratings. Such a rating may be an outlier. Outliers may be identified using ASTM E178-08 (Standard Practice for Dealing with Outlying Observations). Any clearly identified outlier may be removed from the data set.

To provide perspective on the consistency of the panel and to identify any unusual variation in ratings, one of the wipe options presented to the panelists is always a control wipe. The control wipe consists of a specific lotion and substrate combination at a defined lotion load such that the wipe is expected to have a wetness rating close to “just right” on the rating scale. Specifically, the lotion composition is as described in Example 1 above, and the substrate is a commercially available carded spunlaced material consisting of a blend of 60% polypropylene (1.5 denier) and 40% viscose (1.5 denier) fibers with a basis weight of 58 gsm. Such a substrate is available from Suominen of Tampere, Finland as FIBRELLA™ 3160. It is acceptable for the viscose fibers to be replaced with Lyocell fibers of the same denier and percentage of the fiber blend. The lotion and substrate are combined as described above with a specified lotion load of 315%.

If the average sensory rating for the control wipe do not fall within the range of 0 (just right) plus or minus 7, the data for the given sensory study is considered invalid for the purposes of comparing wetness rating between the tested wipe options.

### Rheometry

This method is suitable for determination of the yield value (i.e., the shear stress at which apparent viscosity begins to reduce) of neat cleaning lotions and cleaning lotions expressed from saturated wet wipes.

### Apparatus

| | |
|---|---|
| Rheometer | A Haake model RS600 Rheometer as is |
| | available from Thermo Haake (or |
| | equivalent) of Paramus, NJ is suitable |
| Cone Plate - 60 mm 1° Ti | Haake Part No. 222-1273 |
| Base Plate -for 60 mm sensor | Haake Part No. 222-1298 |
| Constant Temperature Bath | Any model capable of maintaining |
| | 22° ± 1° C. accuracy. |
| Data Acquisition and Analysis | Rheo Win Pro. Version 296 is suitable |
| Software | for operation of the Haake instrument |
| | or equivalent software suitable for |
| | use with instruments should be used. |
| Personal Computer | Any computer capable of running the |
| | RheoWin software |

### Procedure

This procedure is suitable for setup and operation of the Haake instrument. It should be modified as necessary by a skilled rheometer so as to provide equivalent results if another instrument is used.

### Preparation of Equipment

The Haake rheometer geometry and inertia will be calibrated during initial installation and set up by the manufacturer. Consult the owner's manual for further information concerning the operation of both the rheometer and the constant temperature bath.

Equilibrate the constant temperature bath to 22° C.±1° C.

Load the 60 mm base plate by pressing the plate straight down against the spring force of the temperature sensor and turning counter clockwise. This ensures proper seating of the plate. Tighten the threaded connection to attach the 60 mm cone to the rheometer.

Turn on the personal computer and open the RheoWin Pro Job Manager software. Confirm the following setup parameters are entered:

| Parameter | | Value | |
|---|---|---|---|
| a) | Temperature | a) | 22° C. |
| b) | Cont Stress, Stress Sweep Graphic Defn. | b) | |
| c) | Controlled Stress Controlled Rotation | c) | Select: Start τ = 0.0 Pa, End τ = 10.0 Pa, |
| | Ramp | | Distribution = log, Duration = 200 Seconds, |
| | | | Acquisition # data = 400, break shear rate >100 |
| | | | sec⁻¹, |
| d) | Zero Point Measurement Position | d) | Select from Axial Parameters Zero Point Menu: |
| | | | Find and Set Zero Point and Prompt Message |
| | | | When Finished “Load Sample” |
| | | | Select from Axial Parameters/Measurement |
| | | | Position: Go to measurement position, speed- |
| | | | max and, from standby submenu: 2 mm |
| e) | Lift Apart | e) | Select Move Lift Apart from Axial Parameter |
| | | | Sub menu; Enter Prompt “Clean off Sensor and |
| | | | save file” under Axial Options Submenu |
| f) | Devices | f) | 60 mm Cone Plate |
| g) | Display | g) | Mode = Auto |
| h) | Filename | h) | Select: Ask for File Name and At end of job |
| i) | Segments | i) | Select: All Segments under subheading save |
| | | | segments |
| j) | Max Time | j) | 300 seconds |

### Instrument Operation

The job is now properly set up and the rheometer is ready to run. Make sure that the cone plate sensor and the base plate are clean. (isopropanol or ethanol are suitable cleaning aids). Allow the sensor and base plate to dry.

Click Start at the bottom of the job window. The rheometer will zero itself. The base plate will move to reach the parallel plate sensor (top) and automatically zero. When the machine has successfully zeroed itself, a message “Load sample” will appear on the screen.

Using a pipette or equivalent, place approximately 1.15 ml of product on measuring plate. Click OK for load sample. Insure the sample is deposited close to the center of the plate to help assure that the entire gap between the plate and the cone is filled when the apparatus is in a closed configuration.

The rheometer will slowly close. When the gap setting is reached, a message box will appear “Ready to run”. Click OK. The test will now run automatically.

When the test is complete, a message “Test Finished” will appear on the screen. Click OK. The computer will then prompt the operator to “Save as”. Save file appropriately. The rheometer will automatically open. Unscrew the cone plate from the instrument. Clean both the sensor and base plate with a clean lint-free laboratory wipe and isopropanol or ethanol. Allow the cone and base plate to dry before running the next sample.

### Analysis of Data

1. Open the RheoWin Pro Data Manager software. Open the file containing the run data. A data table with information on the side will appear.

2. From the main menu select Layout, Graph Layout, and the Controlled Stress Sweep Template. Click OK. A graph will appear with stress or Tau as the x axis and shear strain on the y axis. If desired, this template may be modified to show apparent viscosity (η) on the y axis.

3. Click the icon at the top of the menu bar which shows τ₀ or yield stress. Select the data and click calculate as new. This yield point evaluation tool performs a curve analysis where the point of deformation or where the curve bends is considered the yield point. The yield point will then be displayed. Alternatively, the intersection of extrapolated line from the relatively horizontal portion of a graph of η vs. τ with a line extrapolated from the declining portion of the graph will define the approximate yield value.

The dimensions and values disclosed herein are not to be understood as being strictly limited to the exact numerical values recited. Instead, unless otherwise specified, each such dimension is intended to mean both the recited value and a functionally equivalent range surrounding that value. For example, a dimension disclosed as “40 mm” is intended to mean “about 40 mm.”

Every document cited herein, including any cross referenced or related patent or application, is hereby incorporated herein by reference in its entirety unless expressly excluded or otherwise limited. The citation of any document is not an admission that it is prior art with respect to any invention disclosed or claimed herein or that it alone, or in any combination with any other reference or references, teaches, suggests or discloses any such invention. Further, to the extent that any meaning or definition of a term in this document conflicts with any meaning or definition of the same term in a document incorporated by reference, the meaning or definition assigned to that term in this document shall govern.

While particular embodiments of the present invention have been illustrated and described, it would be obvious to those skilled in the art that various other changes and modifications can be made without departing from the spirit and scope of the invention. It is therefore intended to cover in the appended claims all such changes and modifications that are within the scope of this invention.

What is claimed is:

1. A wet wipe comprising an aqueous lotion composition releasably carried by a substrate, the lotion composition comprising:
a water soluble superwetter; and
a shear-thinning rheology modifier.

2. The wet wipe of claim 1, wherein the superwetter has an average molecular weight of less than about 3,000.

3. The wet wipe of claim 2, wherein the superwetter has an average molecular weight of less than about 1,500.

4. The wet wipe of claim 2, wherein the superwetter is PEG-8 dimethicone.

5. The wet wipe of claim 3, wherein the superwetter is PEG-8 dimethicone.

6. The wet wipe of claim 1, wherein the superwetter is present at 0.01% to 0.2% weight of the superwetter to weight of the lotion composition.

7. The wet wipe of claim 1, wherein the rheology modifier is present at 0.01% to 0.5% weight of the rheology modifier to weight of the lotion composition.

8. The wet wipe of claim 1, further comprising an emollient and an emulsifier, wherein the lotion composition has a pH equal to or more acidic than 4.5.

9. The wet wipe of claim 8, wherein the emollient is a thickening emollient.

10. The wet wipe of claim 8, wherein the emulsifier is solid at room temperature.

11. The wet wipe of claim 1, further comprising a preservative.

12. The wet wipe of claim 1, further comprising a location load, wherein the lotion load is between 150% and 480%.

13. The wet wipe of claim 1, wherein, for a fixed substrate and wetness sensory rating, the ratio of a lotion load of the lotion of claim 1 to a lotion load of the lotion of Example 1 is 0.6 to 0.9.

14. The wet wipe of claim 1, wherein the superwetter is selected from the group consisting of trisiloxanes; polyether dimethicones wherein the polyether functionality is PEG, PPG, or mixtures of PEG and PPG; and mixtures thereof.

15. The wet wipe of claim 1, wherein the rheology modifier is selected from the group consisting of xanthan gum, modified xanthan gums, and mixtures thereof.

16. The wet wipe of claim 8, wherein the emollient is selected from the group consisting of PEG-10 sunflower oil glycerides; sunflower oil, palm oil, olive oil, emu oil, babassu oil, evening primrose oil, palm kernel oil, cod liver oil, cottonseed oil, jojoba oil, meadowfoam seed oil, sweet almond oil, canola oil, soybean oil, avocado oil, safflower oil, coconut oil, sesame oil, rice bran oil, grape seed oil; mineral oil; isopropyl stearate, isostearyl isononanoate, diethylhexyl fumarate, diisostearyl malate, triisocetyl citrate, stearyl stearate, methyl palmitate, methylheptyl isostearate; petrolatum; lanolin oil and lanolin wax; long chain alcohols like cetyl alcohol, stearyl alcohol, behenyl alcohol, isostearyl alcohol, 2-hexyldecanol; myristyl alcohol; dimethicone fluids of various molecular weights and mixtures thereof; PPG-15 stearyl ether (also known as arlatone E); shea butter; olive butter; sunflower butter; coconut butter; jojoba butter; cocoa butter; squalane and squalene; isoparaffins; polyethylene glycols of various molecular weights; polypropylene glycols of various molecular weights; and mixtures thereof.

17. The wet wipe of claim 8, wherein the emulsifier is selected from the group consisting of laureth-23; ceteth-2; ceteth-10; ceteth-20; ceteth-21; ceteareth-20; steareth-2; steareth-10; steareth-20; oleth-2; oleth-10; oleth-20; steareth-100; steareth-21; PEG-40 sorbitan peroleate; PEG-8 stearate; PEG-40 stearate; PEG-50 stearate; PEG-100 stearate; sorbitan laurate; sorbitan palmitate; sorbitan stearate; sorbitan tristearate; sorbitan oleate; sorbitan trioleate; polysorbate 20; polysorbate 21; polysorbate 40; polysorbate 60; polysorbate 61; polysorbate 65; polysorbate 80; polysorbate 81; polysorbate 85; PEG-40 hydrogenated castor oil (also known as Emulsogen HCW-049); and citric acid ester (also known as Citrem N12 Veg K).

18. The wet wipe of claim 10, wherein the emollient is selected from the group consisting of microcrystalline wax; paraffin wax; beeswax; carnauba wax; ozokerite wax; cetyl alcohol; stearyl alcohol; cetearyl alcohol; myristyl alcohol; behenyl alcohol; and mixtures thereof.

19. A method of increasing the perception of wetness in a wet wipe comprising:
providing a lotion, the lotion comprising a water soluble superwetter and a rheology modifier;
providing a wipe substrate; and
loading the wipe substrate with the lotion.

20. A wet wipe having a wetness sensory rating to lotion load ratio of at least 4.5.
